(19) United States
(12) Patent Application Publication
(10) Pub. No.: US 2014/0328462 A1
(43) Pub. Date: Nov. 6, 2014

(54) MEDICAL IMAGE PROCESSING APPARATUS, X-RAY DIAGNOSTIC APPARATUS, MEDICAL IMAGE PROCESSING METHOD, AND X-RAY DIAGNOSTIC METHOD

(71) Applicants: Kabushiki Kaisha Toshiba, Minato-ku (JP); Toshiba Medical Systems Corporation, Otawara-shi (JP)

(72) Inventors: Hisayuki UEHARA, Otawara-shi (JP); Yoshinori Shimizu, Nasushiobara-shi (JP); Masaki AKIYAMA, Otawara-shi (JP)

(73) Assignees: Kabushiki Kaisha Toshiba, Minato-ku (JP); Toshiba Medical Systems Corporation, Otawara-shi (JP)

(21) Appl. No.: 14/339,481

(22) Filed: Jul. 24, 2014

Related U.S. Application Data
(63) Continuation of application No. PCT/JP2013/067100, filed on Jun. 21, 2013.

(30) Foreign Application Priority Data
Jun. 27, 2012 (JP) 2012-144044

(51) Int. Cl. A61B 6/00

(52) U.S. Cl. CPC A61B 6/5235; A61B 6/541
USPC 378/62

(57) ABSTRACT

According to one embodiment, a medical image processing apparatus includes an image storage part, an image acquisition part, an information addition part and an image composition part. The image storage part stores the first X-ray image data corresponding to different time phases and corresponding to a region including a ROI. The image acquisition part sequentially acquires the second X-ray image data corresponding to the time phases and corresponding to the ROI. The information addition part adds information indicating the time phases to the first and second X-ray image data. The image composition part sequentially combines the first X-ray image data with the second X-ray image data, whose time phase corresponds to a time phase of the first X-ray image data, based on the information indicating the time phases incidental to the first and second X-ray image data.

### CROSS REFERENCES TO RELATED APPLICATIONS

This is a continuation of Application PCT/JP2013/67100, filed on Jun. 21, 2013.

This application is based upon and claims the benefit of priority from Japanese Patent Application No. 2012-144044, filed on Jun. 27, 2012; the entire contents of which are incorporated herein by reference.

### FIELD

Embodiments described herein relate generally to a medical image processing apparatus, an X-ray diagnostic apparatus, a medical image processing method, and an X-ray diagnostic method.

### BACKGROUND

Conventionally, as an image display technology in X-ray imaging, the technology, for storing a still image acquired previously, combining the still image with each X-ray fluoroscopic image acquired in real time under a condition that an X-ray exposure field is narrowed down, and displaying the combined images, is known. Specifically, a still image is used as an image around each X-ray fluoroscopic image acquired as a live image in a condition that an X-ray exposure range is narrowed down. Thereby, it is possible to display live images required for a diagnosis with reducing an X-ray exposure area and a data volume.

### PRIOR TECHNICAL LITERATURE

[Patent literature 1] JPA-H8-164130

[Patent literature 2] JPA2003-265449

When an X-ray image acquired previously is combined with each of X-ray live images acquired in real time for a display, a display with a higher image quality is desired. Similarly, when an X-ray image acquired previously is combined with each of X-ray cine images acquired previously for a display, a display with a higher image quality is desired.

Accordingly, an object of the present invention is to provide a medical image processing apparatus, an X-ray diagnostic apparatus, a medical image processing method, and an X-ray diagnostic method which can combine an X-ray moving image with a precedently acquired X-ray image to display them with a higher image quality.

### BRIEF DESCRIPTION OF THE DRAWINGS

In the accompanying drawings:

FIG. 1 is a configuration diagram of a medical image processing apparatus and an X-ray diagnostic apparatus according to one embodiment of the present invention;

FIG. 2 is a flow chart which shows a flow in case of acquiring and displaying a live image of a heart by the medical image processing apparatus and X-ray diagnostic apparatus shown in FIG. 1;

FIG. 3 is a view for explaining a generation method of live image data to be displayed; and

FIG. 4 is a view showing an example case of indicating also the external of a ROI, displayed as a live image, with intermittent updating.

### DETAILED DESCRIPTION

In general, according to one embodiment, a medical image processing apparatus includes an image storage part, an image acquisition part, an information addition part and an image composition part. The image storage part is configured to store frames of first X-ray image data corresponding to different time phases and corresponding to a region including a region of interest. The image acquisition part is configured to sequentially acquire frames of second X-ray image data corresponding to the time phases and corresponding to the region of the interest. The information addition part is configured to add information indicating the time phases to each of the frames of the first X-ray image data and the frames of the second X-ray image data. The image composition part is configured to combine a frame of the first X-ray image data with a frame of the second X-ray image data, whose time phase corresponds to a time phase of the frame of the first X-ray image data, based on the information indicating the time phases incidental to each of the frames of the first X-ray image data and the frames of the second X-ray image data.

Further, according to another embodiment, an X-ray diagnostic apparatus includes an image storage part, an image acquisition unit, an information addition part and an image composition part. The image storage part is configured to store frames of first X-ray image data corresponding to different time phases and corresponding to a region including a region of interest. The image acquisition unit is configured to sequentially acquire frames of second X-ray image data corresponding to the time phases and corresponding to the region of the interest. The information addition part is configured to add information indicating the time phases to each of the frames of the first X-ray image data and the frames of the second X-ray image data. The image composition part is configured to combine a frame of the first X-ray image data with a frame of the second X-ray image data, whose time phase corresponds to a time phase of the frame of the first X-ray image data, based on the information indicating the time phases incidental to each of the frames of the first X-ray image data and the frames of the second X-ray image data.

Further, according to another embodiment, a medical image processing method includes: storing frames of first X-ray image data corresponding to different time phases and corresponding to a region including a region of interest; sequentially acquiring frames of second X-ray image data corresponding to the time phases and corresponding to the region of the interest; adding information indicating the time phases to each of the frames of the first X-ray image data and the frames of the second X-ray image data; and sequentially combining a frame of the first X-ray image data with a frame of the second X-ray image data, whose time phase corresponds to a time phase of the frame of the first X-ray image data, based on the information indicating the time phases incidental to each of the frames of the first X-ray image data and the frames of the second X-ray image data.

Further, according to another embodiment, an X-ray diagnostic method includes: storing frames of first X-ray image data corresponding to different time phases and corresponding to a region including a region of interest; sequentially acquiring frames of second X-ray image data corresponding to the time phases and corresponding to the region of the interest; adding information indicating the time phases to each of the frames of the first X-ray image data and the frames of the second X-ray image data; and sequentially combining a frame of the first X-ray image data with a frame of the second X-ray image data, whose time phase corresponds to a time phase of the frame of the first X-ray image data, based on the information indicating the time phases incidental to each of the frames of the first X-ray image data and the frames of the second X-ray image data.

A medical image processing apparatus, an X-ray diagnostic apparatus, a medical image processing method, and an X-ray diagnostic method according to embodiments of the present invention will be described with reference to the accompanying drawings.

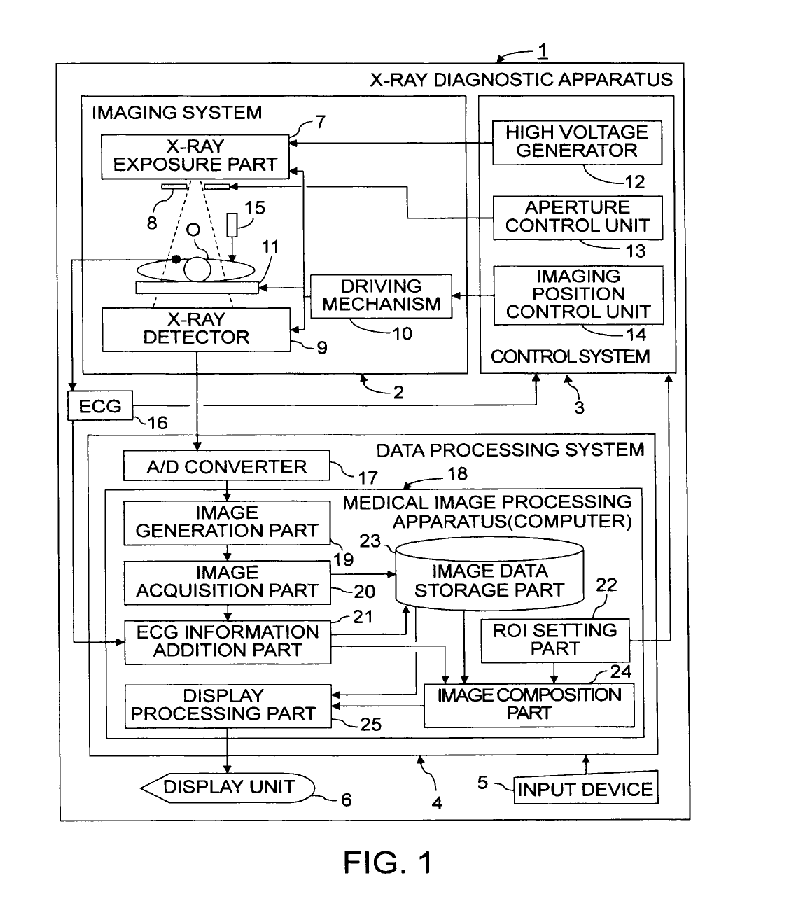

FIG. 1 is a configuration diagram of a medical image processing apparatus and an X-ray diagnostic apparatus according to one embodiment of the present invention.

An X-ray diagnostic apparatus 1 includes an imaging system 2, a control system 3, and a data processing system 4, an input device 5 and a display unit 6 The imaging system 2 has an X-ray exposure part 7, an X-ray adjustable aperture 8, an X-ray detector 9, a driving mechanism 10 and a bed 11. The control system 3 has a high voltage generator 12, an aperture control unit 13, and an imaging position control unit 14.

The X-ray exposure part 7 includes an X-ray tube and is placed in the opposite side of the X-ray detector 9 so that an object O set on the bed 11 lies between the X-ray exposure part 7 and the X-ray detector 9. The X-ray exposure part 7 and the X-ray detector 9 can change the angle and the relative position with respect to the object O with keeping their relative position by driving the driving mechanism 10. Specifically, the X-ray exposure part 7 and the X-ray detector 9 are settled at both ends of the C-shaped arm having the rotational function.

Then, the X-ray exposure part 7 is configured to expose an X-ray from a predetermined angle to an object O by the X-ray tube to detect the X-ray having transmitted the object O by the X-ray detector 9. Further, the X-ray exposure part 7 has the X-ray adjustable aperture 8. Thus, a range of an X-ray exposed from the X-ray tube of the X-ray exposure part 7 towards an object O can be adjusted by the X-ray adjustable aperture 8.

The incline and the position of the table of the bed 11 can be adjusted with the driving mechanism 10. Therefore, the radiation direction of an X-ray toward an object O can be changed by adjusting not only the angle of the X-ray exposure part 7 and the X-ray detector 9 with regard to the object O but also the angle of the table.

Furthermore, a contrast medium injector 15 is provided in the vicinity of the object O set on the bed 11 in order to inject a contrast agent into the object O. Further, an ECG (electrocardiogram) unit 16 for acquiring an ECG signal of the object O is prepared.

The high voltage generator 12 of the control system 3 is a unit which applies a high voltage to the X-ray tube of the X-ray exposure part 7 to expose an X-ray, having a desired energy, toward the object O. The aperture control unit 13 is a unit which adjusts an exposure range of an X-ray by controlling the aperture value of the X-ray adjustable aperture 8. The imaging position control unit 14 is a unit which outputs a control signal to the driving mechanism 10 to control the driving mechanism 10. That is, the inclination and position of the top plate of the bed 11, and the rotation angle and position of the X-ray exposure part 7 and the X-ray detector 9 are controlled by the control signals output to the driving mechanism 10 from the imaging position control unit 14.

Moreover, each element of the control system 3 is configured to operate in synchronized with an ECG signal acquired by the ECG unit 16. Therefore, an X-ray can be exposed and detected in synchronized with an ECG signal.

The data processing system 4 has an A/D (analog to digital) converter 17 and a computer 18. The computer 18 functions as a medical image processing apparatus 18 by executing programs. That is, the medical image processing apparatus 18 is built in the X-ray imaging apparatus 1.

However, an independent medical image processing apparatus having the similar function may be connected to the X-ray imaging apparatus 1 through a network. Moreover, circuits may be used for configuring the medical image processing apparatus 18 built in the X-ray imaging apparatus 1 or the medical image processing apparatus connected with the X-ray imaging apparatus 1 through a network.

The medical image processing apparatus 18 has an image generation part 19, an image acquisition part 20, an ECG information addition part 21, a ROI setting part 22, an image data storage part 23, an image composition part 24, and a display processing part 25.

The image generation part 19 has a function to read digitized X-ray detection data from the X-ray detector 9 through the A/D converter 17 to generate X-ray image data by data processing of the read X-ray detection data. Note that, X-ray contrast image data is to be generated when X-ray detection data is acquired with injecting a contrast agent while X-ray non-contrast image data is to be generated as X-ray fluoroscopic image data when X-ray detection data is acquired without injecting a contrast agent.

Therefore, the collaboration of the data processing system 4 including the image generation part 19 with the imaging system 2 and the control system 3 gives a function as an image acquisition part, configured to acquire X-ray image data of an object O, to the X-ray diagnostic apparatus 1.

The image acquisition part 20 has a function to acquire the X-ray image data generated in the image generation part 19. Especially, in an independent medical image processing apparatus connected to the X-ray imaging apparatus 1 through a network, the image generation part 19 can be omitted. In this case, a function to acquire the X-ray image data from the image generation part 19 included in the X-ray imaging apparatus 1 through a network is provided with the image acquisition part 20.

The ECG information addition part 21 has a function to acquire an ECG signal from the ECG unit 16, and a function to acquire time series frames of X-ray image data from the image acquisition part 20 to add phase information of the ECG signal, corresponding to the respective imaging timings of the frames of the X-ray image data, to the frames of the X-ray image data respectively. A cardiac time phase, added to the X-ray image data, as incidental information can be indicated as a phase angle to a reference wave such as the R wave, or as a delay time from a reference wave such as the R wave. That is, a phase angle to a reference wave of an ECG signal or a delay time from a reference wave of an ECG signal can be added to X-ray image data as information for indicating a cardiac time phase.

The ROI setting part 22 has a function to set up an ROI (region of interest) as an X-ray exposure area according to direction information from the input device 5, and a function to output the set ROI, as information of an X-ray exposure area, to the control system 3. Moreover, the aperture control unit 13 of the control system 3 is configured to control the aperture value of the X-ray adjustable aperture 8 so that the ROI becomes the X-ray exposure area.

The image data storage part 23 is a storage unit which stores the X-ray image data acquired by the image acquisition part 20 and the X-ray image data to which cardiac time phase information has been added by the electrocardiographic information addition part 22.

The image composition part 24 has a function to acquire the first X-ray image data, corresponding to the first ROI, and the second X-ray image data, corresponding to the second ROI, whose cardiac time phase corresponds to that of the first X-ray image data, from the image data storage part 23 or the ECG information addition part 21, to combine the first X-ray image data with the second X-ray image data. The first ROI is set as a display range on the display unit 6. Therefore, the first ROI may be the whole field of view. The second ROI is set as a display range of a live image. Therefore, the second ROI is included in the first ROI. Then, the image composition part 24 is configured to perform image composition processing which replaces the part, corresponding to the second ROI, out of the first X-ray image data, into the second X-ray image data.

The correspondence of cardiac time phases can be determined based on information showing a cardiac time phase added to X-ray image data. For example, when the information showing a cardiac time phase is a phase angle, two frames of X-ray, image data whose phase angles is the same or can be considered to be the same can be acquired as the first and the second X-ray image data whose cardiac time phases correspond to each other, from the image data storage part 23.

When the information showing a cardiac time phase is a delay time from a reference wave, such as an R wave, comparing one delay time with the other delay time may be unsuitable since a period of one heart rate in imaging of the first X-ray image data may differ from that in imaging of the second X-ray image data. Accordingly, the first and second X-ray image data whose cardiac time phases correspond to each other can be specified by multiplying a ratio, between periods of one heart rates during which the first and second X-ray image data have been acquired respectively, with a delay time added to one of the first and second X-ray image data.

The display processing part 25 has a function to acquire X-ray image data, to be a display target on the display unit 6, from the image composition part 24 or the image data storage part 23, to perform necessary display processing such as gradation processing and spatial filter processing, and also has a function to display the X-ray image data after the display processing on the display unit 6.

Next, an operation and an action of the medical image processing apparatus 18 and the X-ray diagnostic apparatus 1 will be explained.

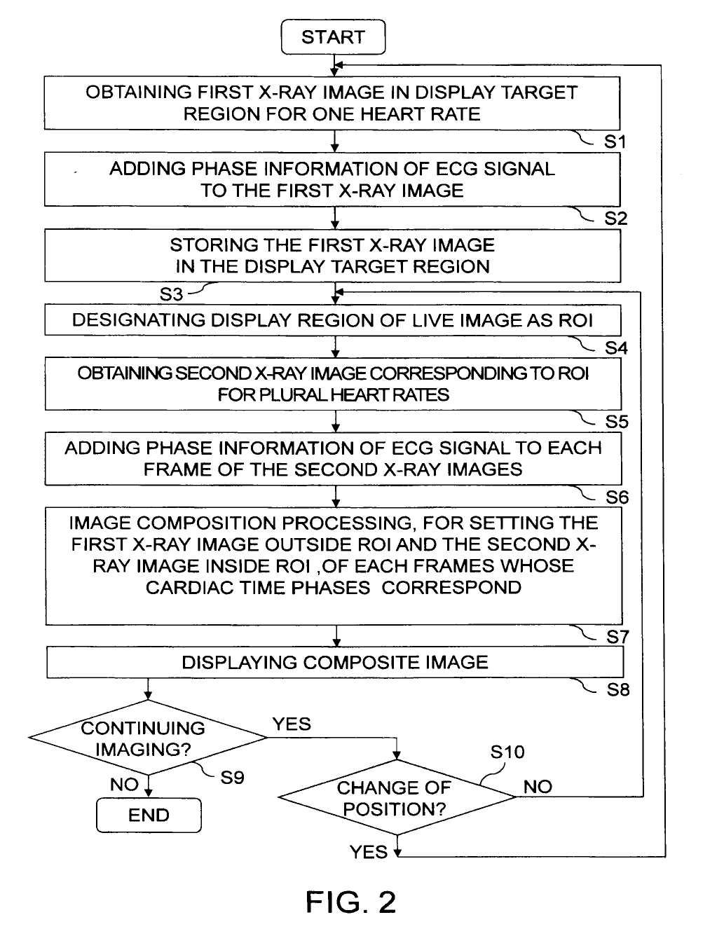

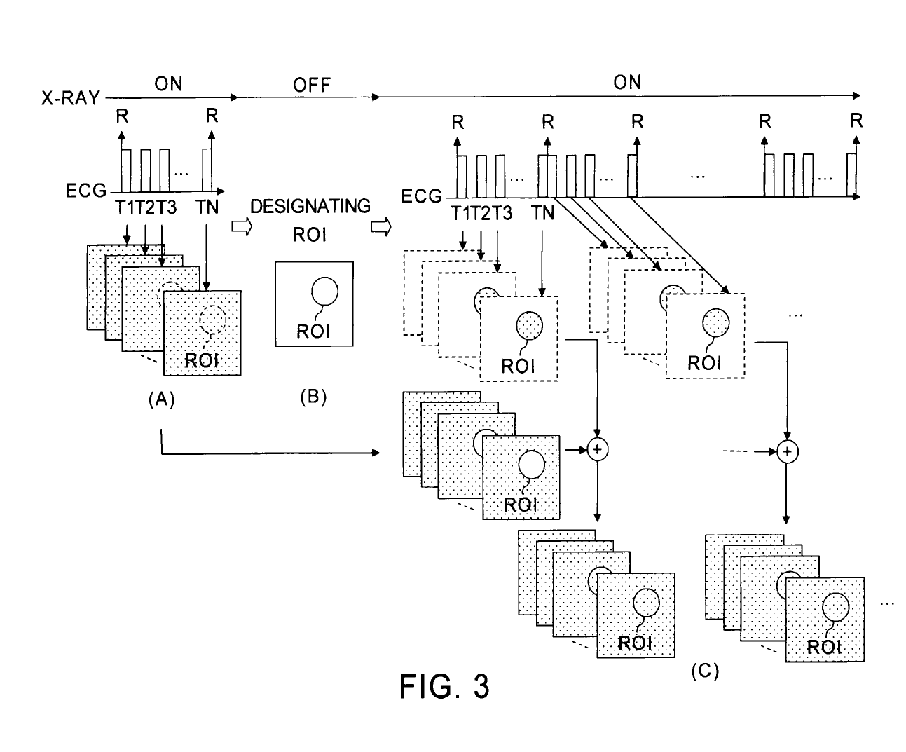

FIG. 2 is a flow chart which shows a flow in case of acquiring and displaying a live image of a heart by the medical image processing apparatus 18 and X-ray diagnostic apparatus 1 shown in FIG. 1 Moreover, FIG. 3 is a view for explaining a generation method of live image data to be displayed.

At first, in Step S1, X-ray moving image data for one heart rate are acquired from a region which covers at least a region including a heart as a display target of diagnostic images. That is, frames of the first X-ray image data corresponding to plural cardiac time phases for one heart rate are acquired. A contrast agent is administered, as needed, and moving image data are acquired as X-ray contrast image data. Alternatively, X-ray fluoroscopic image data are acquired as moving image data without an administration of a contrast agent.

More specifically, information which directs an imaging region is input from the input device 5 to the ROI setting part 22. The imaging region is set to include a surrounding region of an ROI set as a display region of a live image. Accordingly, the whole field of view is set as the imaging region, for example. Then, the information for directing to image the whole field of view is output from the ROI setting part 22 to the control system 3.

Next, control signals according to the imaging conditions are output from the imaging position control unit 14 of the control system 3 to drive the driving mechanism 10. Thereby, the X-ray exposure part 7 and the X-ray detector 9 are positioned to a predetermined position. Further, the aperture control unit 13 controls the aperture degree of the X-ray adjustable aperture 8 to become the maximum. Thereby, the whole field of view is set to an exposed area by an X-ray.

On the other hand, a high voltage is applied to the X-ray tube of the X-ray exposure part 7 from the high voltage generator 12 of the control system 3. Thereby, an X-ray is exposed to an imaging part of the object O from the X-ray tube. Then, the X-ray which transmitted the object O is detected by the X-ray detector 9. Note that, the X-ray exposure is performed in synchronized with an ECG signal acquired by the ECG unit 16. That is, the control system 3 controls the imaging system 2 in synchronized with an ECG signal.

Next, an X-ray detection signal is output to the medical image processing apparatus 18 from the X-ray detector 9 through the A/D converter 17. Thereby, the digitized X-ray detection data is acquired in the image generation part 19. Then, the image generation part 19 generates X-ray image data by known data processing of the X-ray detection data. The X-ray image data generated in the image generation part 19 is given to the image acquisition part 20. Thereby, the image acquisition part 20 acquires frames of the first X-ray image data, corresponding to cardiac time phases for one heart rate, of the whole field of view.

Next, in step S2, the ECG information addition part 21 acquires an ECG signal from the ECG unit 16, and adds the phase information of the ECG signal to the frames of the first X-ray image data as incidental information showing the cardiac time phases, respectively.

Next, in Step S3, the ECG information addition part 21 writes and store the first X-ray image data, to which the phase information of the ECG signal has been added, in the image data storage part 23. As a result, the frames of the first X-ray image data respectively corresponding to the different plural cardiac time phases T1, T2, T3, . . . , TN and each corresponding to the region including the ROI for the live image as shown in FIG. 3(A) are stored in the image data storage part 23. Moreover, the information indicating the cardiac time phases T1, T2, T3, . . . , TN are added to the frames of the first X-ray image data respectively as incidental information.

Next, in Step S4, specification information of the ROI is input from the input device 5 into the ROI setting part 22 as a display region of the live image. Then, the ROI setting part 22 sets up the ROI according to the information input from the input device 5, as the display region of the live image as shown in FIG. 3(B). The ROI setting part 22 notifies each of the control system 3 and the image composition part 24 of the set ROI.

Next, in Step S5, live image data of the set ROI is acquired as moving image data in synchronization with the ECG. That is, the second X-ray image data corresponding to the ROI are acquired in a flow similar to that in the acquisition of the first X-ray image data. However, the aperture value of the X-ray adjustable aperture 8 is controlled by the aperture control unit 13 so that an exposed area by an X-ray is narrowed down into inside of the ROI. As a result, frames of the second X-ray image data respectively corresponding to plural cardiac time phases and each corresponding to the ROI are acquired sequentially in the image acquisition part 20. Note that, the second X-ray image data can be acquired during a required period, as frames of X-ray image data corresponding to plural heart rates.

Next, in Step S6, the ECG information addition part 21 acquires an ECG signal from the ECG unit 16 to sequentially add phase information of the ECG signal, as information indicating cardiac time phases, to the corresponding frames of the second X-ray image data, as incidental information.

Next, in Step S7, the image composition part 24 sequentially acquires the time series frames of the second X-ray image data, to which phase information of the ECG signal are added, from the ECG information addition part 21. On the other hand, the image composition part 24 sequentially acquires the frames of the first X-ray image data, whose cardiac time phases correspond to the cardiac time phases of the frames of the second X-ray image data, from the image data storage part 23.

Then, the image composition part 24 sequentially combines the frames of the first X-ray image data with the frames of the second X-ray image data, whose cardiac time phases correspond to those of the frames of the first X-ray image data, based on information indicating plural cardiac time phases added to each of the first and second X-ray image data. Specifically, the image composition part 24 performs image composition processing which replaces the part corresponding to the ROI for the live image, out of the first X-ray image data, with the second X-ray image data whose cardiac time phases are same. As a result, time series frames of X-ray image data for display, whose pixel values inside the ROI are those of the second X-ray image data and pixel values outside the ROI are those of the first X-ray image data, are sequentially generated as shown in FIG. 3 (C).

Next, in Step S8, the display processing part 25 sequentially acquires the frames of the X-ray composite image data for display from the image composition part 24 to perform necessary display processing of the frames of the X-ray composite image data for display to output the frames of the X-ray composite image data for display to the display unit 6. As a result, composite images, whose regions outside the ROI are the previously acquired first X-ray images and regions inside the ROI are the second X-ray images acquired in real time, are dynamically displayed as a moving image on the display unit 6.

Consequently, a user can observe the second images whose peripheral regions are the first images and each depicting a diagnostic part in real time. Moreover, the X-ray images can be displayed by exposing X-rays to a region which is narrower than the X-ray exposure range corresponding to the region of the X-ray images displayed on the display unit 6. Furthermore, the first images, displayed as peripheral images of the second images displayed in real time, have the same cardiac time phases as those of the second images. Therefore, the continuity between the first images and the second images is sufficient even when a motion caused by beats occurs in a display region.

Next, in Step S9, the data processing system 4 determines whether an instruction for imaging continuation has been input from the input device 5. Note that, whether an instruction for imaging completion has been input, like whether an imaging completion button has been pushed, may be determined. When an instruction for imaging continuation has not been input or an instruction for imaging completion has been input, a series of imaging of the object O is completed.

On the contrary, when an instruction for imaging continuation has been input from the input device 5 into the data processing system 4 or an instruction for imaging completion has not been input from the input device 5 into the data processing system 4, the data processing system 4 waits for an input of information indicating whether a position of the object O has been changed or an instruction for imaging of the first X-ray image data, from the input device 5, in Step S10.

The information indicating whether a position of the object O has been changed may be an instruction, such as an instruction for imaging of the first X-ray image data, according to an existence of a positional change in the object O. Moreover, an instruction for imaging of the first X-ray image data may be also input as an instruction for changing an imaging region, from the input device 5 into the control system 3 through the ROI setting part 22 of the data processing system 4, regardless of an existence of a positional change in the object O.

That is, when a position of the object O has been changed or when an imaging region is changed, an instruction for imaging of the first X-ray image data can be input from the ROI setting part 22 into the control system 3, as a direction of an imaging region. Moreover, an imaging direction of the new first X-ray image data can be input with setting an imaging region same as or different from the imaging region of the first X-ray image data acquired in the past.

Therefore, when the imaging direction of the first X-ray image data, whose imaging region is the X-ray exposure field, has not been input from the input device 5 into the ROI setting part 22, the data processing system 4 determines that a position of the object O has not changed. For this reason, the medical image processing apparatus 18 and the X-ray diagnostic apparatus 1 perform the operations and the processing from Step S4 again. That is, the ROI for the live display can be updated in real time with continuing the X-ray imaging. However, the specification of the ROI as the display region for the live images in Step S4 may be skipped when the ROI does not change.

On the other hand, when the imaging direction of the first X-ray image data, whose imaging region is the X-ray exposure field, has been input from the input device 5 into the ROT setting part 22, the data processing system 4 determines that a position of the object O or the imaging region has changed. For this reason, the medical image processing apparatus 18 and the X-ray diagnostic apparatus 1 perform the operations and the processing from Step S1 again.

That is, whenever a position of the object O or the imaging region has changed, the operations and the processing from Step S1 can be performed. In this case, the X-ray imaging including switches between on and off of an X-ray exposure as shown in the example of FIG. 3 is to be repeated plural times. On the other hand, when a position of the object O or the imaging region has not changed, the moving image can be displayed, with setting the ROI repeatedly as needed, in the condition that the X-ray exposure has been turned on as mentioned above.

In the above-mentioned example, a case of displaying the second images as live images has been explained. However, the first image data and the second image data may be stored in the image data storage part 23 so that the second image data can be combined with the first image data to display the second image data as cine image data.

That is, the medical image processing apparatus 18 and X-ray diagnostic apparatus 1 mentioned above are configured to previously acquire moving image data for one heart rate as image data for a region surrounding an ROI, and to combine the previously acquired moving image data with moving image data in the RO1 for a diagnostic target, in synchronization with corresponding phases of an ECG signal, for display.

For this reason, according to the medical image processing apparatus 18 and the X-ray diagnostic apparatus 1, a moving image of a region including a surrounding region of an ROI can be displayed by only acquiring moving image data in the ROI. Therefore, an exposure dose of the object O can be reduced. Moreover, cardiac time phases of moving image data inside an ROI correspond to those of moving image data outside the ROI to be combined. For this reason, the continuity of internal organs can be maintained on a displayed moving image even when a motion arises in a display region of the image due to the heartbeat. That is, a moving image can be displayed with dynamically following a motion of an internal organ such as a heart.

For example, when a single frame of image data is used as still image data in a peripheral region by the LIH (Last Image Hold) function, which stores the last frame of image data out of time series X-ray fluoroscopic image data, a position gap arises between live image data, in which positions of internal organs change every moment due to motions by heartbeat, and the still image data for the peripheral region. In a typical example, position gaps arise at most phases during one heart rate and disappear at only specific phases.

On the contrary, according to the medical image processing apparatus 18 and the X-ray diagnostic apparatus 1, frames of image data are combined with other frames of image data at corresponding cardiac time phases. For this reason, a moving image without position gaps can be displayed. Therefore, a user can grasp a positional relationship of internal organs, such as a heart, more correctly by observing the moving image. Moreover, exposure doses of both a user and the object O can be reduced by frequently using the image composite function as mentioned above.

Furthermore, the operation can be simplified by performing an acquisition and storage of a moving image for a peripheral region outside an ROI for a live image, as a part of an examination, as shown in the examples of FIG. 2 and FIG. 3.

While certain embodiments have been described, these embodiments have been presented by way of example only, and are not intended to limit the scope of the inventions. Indeed, the novel methods and systems described herein may be embodied in a variety of other forms; furthermore, various omissions, substitutions and changes in the form of the methods and systems described herein may be made without departing from the spirit of the inventions. The accompanying claims and their equivalents are intended to cover such forms or modifications as would fall within the scope and spirit of the inventions.

For example, a case of X-ray imaging of a heart with an ECG synchronization has been described in the above-mentioned example. However, X-ray synchronization imaging may be performed using another synchronization signal, such as a PPG (peripheral pulse gating) signal. A PPG signal is a pulse wave detected as an optical signal from a fingertip or the like. In the case of acquiring a PPG signal, a PPG signal detection unit is installed.

Moreover, in the case of imaging an abdominal region, X-ray imaging can be performed in synchronization with breathing phases. A breathing synchronization signal can be acquired from an arbitrary sensor, such as a flow sensor for detecting breathing, a pressure sensor, or a position sensor installed at an abdominal part.

Then, frames of the first X-ray image data corresponding to different plural time phases and a region including a ROI can be stored in the image data storage part 23. On the other hand, frames of the second X-ray image data corresponding to plural time phases and the ROI can be sequentially acquired in the image acquisition part 20. Furthermore, the frames of the first X-ray image data can be sequentially combined with the frames of the second X-ray image data whose time phases correspond to those of the first X-ray image data, in the image composition part 24, based on the information added to each of the first and second X-ray image data and indicating plural time phases, such as plural cardiac time phases or breathing phases.

Further, in the above-mentioned example, the first X-ray image data, which are not displayed as a live image, are not to be updated as long as a position of the object O and an imaging region do not change. However, the first X-ray image data may be also updated intermittently.

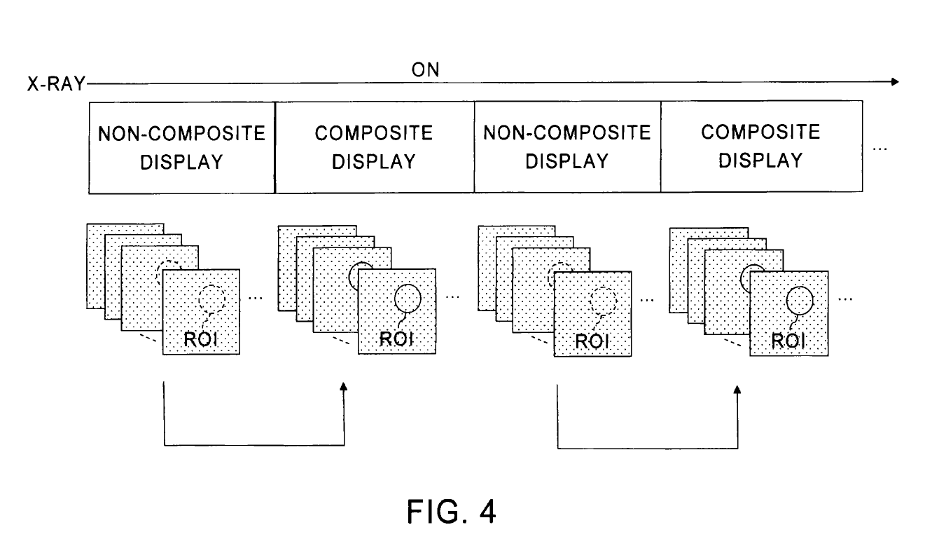

FIG. 4 is a view showing an example case of indicating also the external of a ROI, displayed as a live image, with intermittent updating.

As shown in FIG. 4, imaging the first X-ray image data in an imaging region including an ROI for a live display to display the first X-ray image data without composition and displaying the first X-ray image data combined with the second X-ray image data in the ROI for the live display can be alternately repeated.

In this case, the image acquisition unit including the data processing system 4, the imaging system 2, and the control system 3 is configured to acquire the frames of the new first X-ray image data corresponding to the region including the ROI when a trigger has been acquired. The trigger for starting an acquisition of the new first X-ray image data can be arbitrary information. For example, a predetermined elapsed time or direction information input from the input device 5 into the image acquisition unit can be set as a trigger for starting an acquisition of the new first X-ray image data.

When a predetermined elapsed time is set as a trigger, an acquisition of the first X-ray image data can be automatically and periodically repeated. On the other hand, when a direction information input from the input device 5 into the image acquisition unit is set as a trigger, an acquisition of the first X-ray image data can be started manually at a desired timing.

The new first X-ray image data is acquired during at least one heart rate. When an acquisition of the new first X-ray image data has been completed, the image acquisition unit is configured to acquire the second X-ray image data until the image acquisition unit acquires the next trigger

The first X-ray image data to be acquired again may be acquired during not less than one heart rate. In this case, an acquisition of the second X-ray image data can be started by another trigger. Therefore, in the case where an acquisition of the first X-ray image data and an acquisition of the second X-ray image data are periodically repeated, the image acquisition unit is configured to alternately and repeatedly perform an acquisition of the new first X-ray image data using the first predetermined elapsed time as the first trigger and an acquisition of the second X-ray image data using the second predetermined elapsed time as the second trigger.

On the other hand, the image composition part 24 is configured to sequentially combine the frames of the new first X-ray image data with the frames of the second X-ray image data whose time phases correspond to those of the new first X-ray image data, based on the information, indicating plural time phases, added to each of the new first X-ray image data and the second X-ray image data when the second X-ray image data have been acquired following the new first X-ray image data. In the case where the first X-ray image data and the second X-ray image data are acquired repeatedly, it is suitable to set the first X-ray image data, acquired just before the second X-ray image data, as a composition target.

Then, the display processing part 25 can alternately display the new first X-ray image data and the composite image data, between the new first X-ray image data and the second X-ray image data, on the display unit 6. By such a refreshing function outside the ROI, the background of the ROI can be also updated periodically. For this reason, it becomes possible to grasp a motion in the background of the ROI, a flow of a contrast agent in the background region, and so on.

What is claimed is:

1. A medical image processing apparatus comprising:
an image storage part configured to store frames of first X-ray image data corresponding to different time phases and corresponding to a region including a region of interest;
an image acquisition part configured to sequentially acquire frames of second X-ray image data corresponding to the time phases and corresponding to the region of the interest;
an information addition part configured to add information indicating the time phases to each of the frames of the first X-ray image data and the frames of the second X-ray image data; and
an image composition part configured to combine a frame of the first X-ray image data with a frame of the second X-ray image data, whose time phase corresponds to a time phase of the frame of the first X-ray image data, based on the information indicating the time phases incidental to each of the frames of the first X-ray image data and the frames of the second X-ray image data.

2. A medical image processing apparatus of claim
wherein said information addition part is configured to add information indicating cardiac time phases to each of the frames of the first X-ray image data and the frames of the second X-ray image data, as the information indicating the time phases.

3. A medical image processing apparatus of claim
wherein said information addition part is configured to add phase angles, from a reference wave of an electrocardiogram signal, to each of the frames of the first X-ray image data and the frames of the second X-ray image data, as the information indicating the cardiac time phases.

4. A medical image processing apparatus of claim 2,
wherein said information addition part is configured to add delay times, from a reference wave of an electrocardiogram signal, to each of the frames of the first X-ray image data and the frames of the second X-ray image data, as the information indicating the cardiac time phases.

5. A medical image processing apparatus of claim 4,
wherein said image composition part is configured to specify a frame of the first X-ray image data whose cardiac time phase corresponds to a cardiac time phase of a frame of the second X-ray image data by multiplying a delay time, incidental to one of the frame of the first X-ray image data and the frame of the second X-ray image data, with a ratio between a period of one heart rate during which the frames of the first X-ray image data have been acquired and a period of one heart rate during which the frames of the second X-ray image data have been acquired.

6. A medical image processing apparatus of claim 1,
wherein said information addition part is configured to add information indicating breathing phases to each of the frames of the first X-ray image data and the frames of the second X-ray image data, as the information indicating the time phases.

7. An X-ray diagnostic apparatus comprising:
an image storage part configured to store frames of first X-ray image data corresponding to different time phases and corresponding to a region including a region of interest;
an image acquisition unit configured to sequentially acquire frames of second X-ray image data corresponding to the time phases and corresponding to the region of the interest;
an information addition part configured to add information indicating the time phases to each of the frames of the first X-ray image data and the frames of the second X-ray image data; and
an image composition part configured to combine a frame of the first X-ray image data with a frame of the second X-ray image data, whose time phase corresponds to a time phase of the frame of the first X-ray image data, based on the information indicating the time phases incidental to each of the frames of the first X-ray image data and the frames of the second X-ray image data.

8. An X-ray diagnostic apparatus of claim 7,
wherein said image acquisition unit is configured to acquire frames of new first X-ray image data corresponding to the region including the region of the interest when a trigger has been acquired; and
said image composition part is configured to combine a frame of the new first X-ray image data with the frame of the second X-ray image data whose time phase correspond to a time phase of the new first X-ray image data, based on the information indicating the time phases incidental to each of the frames of the new first X-ray image data and the frames of the second X-ray image data when the frames of the second X-ray image data have been acquired following the frames of the new first X-ray image data.

9. An X-ray diagnostic apparatus of claim 8,
wherein said image acquisition unit is configured to acquire the frames of the new first X-ray image data using a predetermined elapsed time as the trigger.

10. An X-ray diagnostic apparatus of claim
wherein said image acquisition unit is configured to acquire the frames of the new first X-ray image data using instruction information, input from an input device, as the trigger.

11. An X-ray diagnostic apparatus of claim 8,
wherein said image acquisition unit is configured to alternately and repeatedly perform an acquisition of the frames of the new first X-ray image data using a first predetermined elapsed time as a first trigger and an acquisition of the frames of the second X-ray image data using a second predetermined elapsed time as a second trigger,
further comprising:
a display processing part configured to alternately display the frames of the new first X-ray image data and frames of composite image data between the frames of the new first X-ray image data and the frames of the second X-ray image data.

12. A medical image processing method comprising:
storing frames of first X-ray image data corresponding to different time phases and corresponding to a region including a region of interest;
sequentially acquiring frames of second X-ray image data corresponding to the time phases and corresponding to the region of the interest;
adding information indicating the time phases to each of the frames of the first X-ray image data and the frames of the second X-ray image data; and
sequentially combining a frame of the first X-ray image data with a frame of the second X-ray image data, whose time phase corresponds to a time phase of the frame of the first X-ray image data, based on the information indicating the time phases incidental to each of the frames of the first X-ray image data and the frames of the second X-ray image data.

13. An X-ray diagnostic method comprising:
storing frames of first X-ray image data corresponding to different time phases and corresponding to a region including a region of interest;
sequentially acquiring frames of second X-ray image data corresponding to the time phases and corresponding to the region of the interest;
adding information indicating the time phases to each of the frames of the first X-ray image data and the frames of the second X-ray image data; and
sequentially combining a frame of the first X-ray image data with a frame of the second X-ray image data, whose time phase corresponds to a time phase of the frame of the first X-ray image data, based on the information indicating the time phases incidental to each of the frames of the first X-ray image data and the frames of the second X-ray image data.
